(19) United States
(12) Patent Application Publication
(10) Pub. No.: US 2011/0315145 A1
(43) Pub. Date: Dec. 29, 2011

(54) INFANT FRIENDLY NASAL CPAP CANULA SEAL

(76) Inventors: Timothy R. Beevers, McMinnville, OR (US); Katherine K. Beevers, McMinnville, OR (US)

(21) Appl. No.: 12/824,972

(22) Filed: Jun. 28, 2010

(51) Int. Cl. A61M 16/06

(52) U.S. Cl. 128/207.13

(57) ABSTRACT

A nasal CPAP canula seal having directions of “above” and “below” by its anticipated placement on an infant face, above being higher up on the face and below being lower down. The canula seal includes a canula supporting portion, defining apertures for canula passage into the nostrils, and an anchoring portion designed to help anchor the nasal CPAP canula seal by adhering to facial areas about the nostrils. The anchoring portion defines a cut-out centered below the apertures, to help avoid covering any portion of the vermillion of the upper lip with the anchoring portion.

### BACKGROUND

Continuous positive airway pressure is a method of sustaining the breathing of fragile infants (typically prematurely born infants) by gently blowing air into the nostrils to maintain positive pressure in the airways, thereby keeping the airways open. Canula seals that gently adhere to the infants nose, cheek and upper part of the upper lip, and that provide a pair of apertures for supporting the canulas that are placed into the infant nostrils to deliver the air have been commercially available. Also, canula seals have been made by neo-natal nurses by cutting a pattern from wound dressing. Those using these canula seals have noticed some phenomena that have not been 100% desirable. Sometimes a portion of the canula seal would peel away from the infant's face. Also, health care personnel would sometimes place some of the portion intended to cover the philtrum over the vermillion of the infant's upper lip.

In a different environment, these issues might have escaped notice entirely, but when dealing with premature infants, even the smallest issue draws attention. Accordingly, careful study has identified the source of these problems and to recognition of the desirability of design improvements.

### SUMMARY

The following embodiments and aspects thereof are described and illustrated in conjunction with systems, tools and methods which are meant to be exemplary and illustrative, not limiting in scope. In various embodiments, one or more of the above-described problems have been reduced or eliminated, while other embodiments are directed to other improvements.

In a first separate aspect, the present invention may take the form of a nasal CPAP canula seal having directions of “above” and “below” by its anticipated placement on an infant face, above being higher up on the face and below being lower down. The canula seal includes a canula supporting portion, defining apertures for canula passage into the nostrils, and an anchoring portion designed to help anchor the nasal CPAP canula seal by adhering to facial areas about the nostrils. The anchoring portion defines a cut-out centered below the apertures, to help avoid covering any portion of the vermillion of the upper lip with the anchoring portion.

In a second separate aspect, the present invention may take the form of a nasal CPAP canula seal having directions of “above” and “below” by its anticipated placement on an infant face, above being higher up on the face and below being lower down. The seal includes a canula supporting portion, defining two apertures for canula passage into the nostrils, the apertures defining a aperture-spacing length, the length being the distance between the centers of the two apertures, and a nose-anchoring portion extending upwardly from the apertures and designed to help anchor the nasal CPAP canula seal by adhering to the infant nose. The nose-anchoring portion extends no more than 2 aperture-spacing lengths away from the closest of the two apertures.

In a third separate aspect, the present invention may take the form of a nasal CPAP canula seal having directions of “above” and “below” by its anticipated placement on an infant face, above being higher up on the face and below being lower down. The seal includes a canula supporting portion, defining two apertures for canula passage into the nostrils, and a nose-anchoring portion, having a shape and extending upwardly from the apertures and designed to help anchor the nasal CPAP canula seal by adhering to the infant nose. The shape of the nose-anchoring portion includes no acute angles.

In addition to the exemplary aspects and embodiments described above, further aspects and embodiments will become apparent by reference to the drawings and by study of the following detailed descriptions.

### BRIEF DESCRIPTION OF THE DRAWINGS

Exemplary embodiments are illustrated in referenced drawings. It is intended that the embodiments and figures disclosed herein are to be considered illustrative rather than restrictive.

FIG. 1 is a plan view of an infant nasal CPAP canula seal according to the present invention.

FIG. 2 is a perspective view of the seal of FIG. 1 in place on an infant's face.

FIG. 3 is a perspective view of the seal of FIG. 1 in place on an infant's face, supporting a CPAP canula.

### DETAILED DESCRIPTION OF THE PREFERRED EMBODIMENT

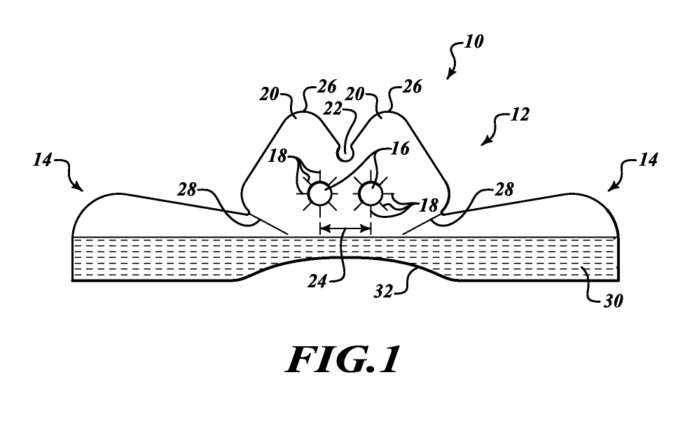

Referring to FIG. 1, a preferred embodiment of an infant nasal CPAP canula seal 10. Seal 10 is die-cut from a sheet of wound dressing material made of hydrocolloid laminated to a 25 micrometer thick sheet of polyurethane. The hydrocolloid gently adheres to the infant face. Seal 10 is sold in a clean package, and on a sheet of backing material, from which it is removed, prior to use.

In alternative preferred embodiments, the layer of seal 10 that contacts the infant face is made of silicone or acrylic adhesive. For example, Silbione 4512 A/B, which may be obtained from a distributor named on the website www.bluestarsilicones.com may be used. One advantage of this type of material is that it can be washed and yet still maintain its adherent properties. Skilled persons will recognize that the sheet of polyurethane may be replaced with any one of a broad range of biocompatible films that are widely available and many of which are clear. If both materials used are transparent, the seal 10 has an additional advantage, that the infant skin tissue will be more clearly visible underneath seal 10. This will help medical personnel quickly spot potential problems.

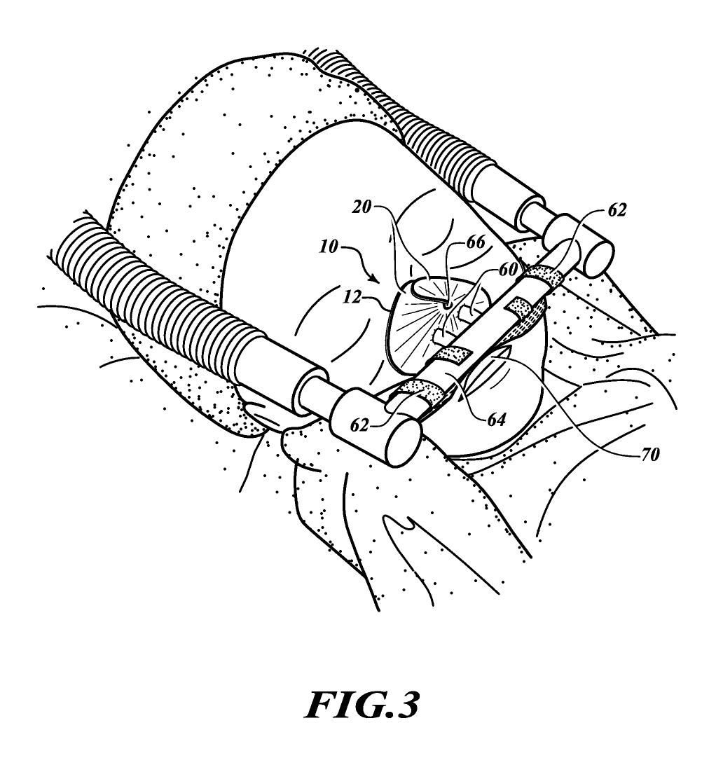

Seal 10 includes a nose covering portion 12 and a lip and cheek adhering portion 14. Nose covering portion 12 defines a pair of nostril apertures 16 for supporting the CPAP tubes 60 (FIG. 3) as they enter the nostrils. Radially emanating cuts or stellations 18, permit the resultant flaps to separate and protect the nostril interiors from direct contact with rigid tubes 60.

The nose covering portion 12 includes two petals 20 and defines a notch 22. In the use of prior art canula seals, it was found that during application of the seal, petals would sometime approach the eye of the infant. After analysis and experimentation, it was found that shorter rounded petals still provided sufficient adhesion and yet avoided this problem.

Apertures 16 define a center-to-center spacing length 24. It has been found that, across the various sizes seals 10, that if the length of petals 20 is kept below two center-to-center spacing lengths 24, that the danger of approaching the eye is minimized.

The acute angles of prior art seals appear to have provided a point for seal 10 to begin to separate from the skin to which it was adhered. It has been found that one way to avoid this problem is to avoid acute angles at the tips 26 of petals 20, in favor, ideally of an arcuate upper edge at tips 26. The definition of nose covering portion 12 is completed by a pair of cuts 28, permitting portion 12 to separate from portion 14 during application.

The bottom portion of lip and cheek adhering portion 14 is covered with hook material tape 30, that is applied during manufacturing and has outwardly projecting hooks. Referring to FIG. 3, loop material 62, wrapped about horizontal tube 64 sticks to this hook material 30, to keep the horizontal tube 64 and therefore the nose tubes 60 in place.

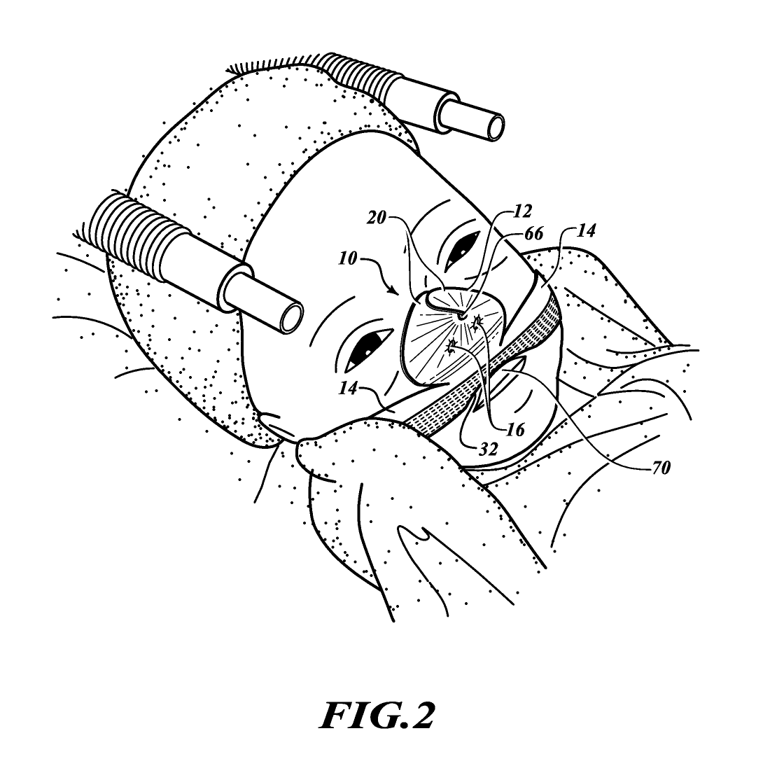

Lip and cheek portion 14 defines a cut-out 32, which helps medical personnel avoid placing a portion of the seal 10 on the vermillion 70 (FIGS. 2+3) of the upper lip. The vermillion is the red part of the lip, as opposed to the thicker-skinned portion that extends upwardly toward the nose and cheek. The vermillion 70 is very sensitive and ideally should not covered. Some have suggested that covering a portion of the vermillion 70 may interfere with an infants ability to begin nursing. Whether or not this is accurate, cutout 32 makes it easier to avoid placing a portion of seal 10 over the vermillion 70, to avoid any resulting problems, and to provide greater infant comfort.

As noted previously petals 20 define a notch 22. When applied about the infant nose, as shown in FIGS. 2 and 3, with one petal 20 folded onto another, this provides a vent 66 for condensation to escape. It had been noticed that the view of the nose afforded to medical personnel was sometimes obscured by cloudiness on the inner surfaces of petals 20. It is desirable that an unobscured view of the nose be available, so that medical personnel can monitor the condition of nose tissue. After analysis and experimentation it was determined that this cloudiness was caused by condensation that had no avenue of escape and built up as a layer on the inner surfaces of petals 20.

This condensation also served to loosen the bond between the nose covering portion 12 and the underlying nose. By providing notch 22 and thereby vent 66 water vapor is permitted to escape and visibility of the nose is maintained. Also, the adhesive bond between nose covering portion 12 and nose is less vulnerable to attack by condensation.

Placement of the nostril apertures 16 is an exercise in delicate determination of a difficult to optimize location. To accommodate all nose shapes (representative of different racial groups and variations within racial groups) it has been found that optimally, even without the cut-out 32, it is important that apertures 16 be close to the bottom of lip and cheek adhering portion 14. Generally speaking, it is desirable that this distance be no greater than two center-to-center spacing lengths 24, and optimally less than one and one-half center-to-center spacing lengths 24.

While a number of exemplary aspects and embodiments have been discussed above, those possessed of skill in the art will recognize certain modifications, permutations, additions and sub-combinations thereof. It is therefore intended that the following appended claims and claims hereafter introduced are interpreted to include all such modifications, permutations, additions and sub-combinations as are within their true spirit and scope.

1. A nasal CPAP canula seal having directions of “above” and “below” by its anticipated placement on an infant face, above being higher up on said face and below being lower down, comprising:
(a) a canula supporting portion, defining apertures for canula passage into the nostrils;
(b) an anchoring portion designed to help anchor said nasal CPAP canula seal by adhering to facial areas about the nostrils; and
(c) wherein said anchoring portion defines a cut-out centered below said apertures, to help avoid covering any portion of the vermillion of the upper lip with said anchoring portion.

2. The canula seal of claim 1, wherein said cut-out is crescent shaped.

3. The canula seal of claim 1, wherein said apertures define a center-to-center length and said cut-out defines a minimum distance from one of said apertures and said maximum value of said minimum distance is 1.5 center-to-center lengths.

4. The canula seal of claim 1, wherein said anchoring portion includes side extensions each extending sideways from about 2 to 3 cm from a center defined at about 0.5 cm below a point exactly midway between the two apertures.

5. The canula seal of claim 1, wherein said side extensions are covered over their side extents by a non stretch material.

6. The canula seal of claim 5, wherein said side extensions have vertical extents from upper to lower borders and wherein said vertical extents are not entirely covered by said non stretch material.

7. The canula seal of claim 5, wherein said non stretch material is hook material.

8. The canula seal of claim 5, wherein said non stretch material is loop material.

9. The canula seal of claim 1, made of a sheet of hydrocolloid laminated to polyurethane.

10. The canula seal of claim 1, defining a set of cuts emanating radially from said apertures.

11. A nasal CPAP canula seal having directions of “above” and “below” by its anticipated placement on an infant face, above being higher up on said face and below being lower down, comprising:
(a) a canula supporting portion, defining two apertures for canula passage into the nostrils, said apertures defining a aperture-spacing length, said length being the distance between the centers of said two apertures;
(b) a nose-anchoring portion extending upwardly from said apertures and designed to help anchor said nasal CPAP canula seal by adhering to the infant nose; and
(c) wherein said nose-anchoring portion extends no more than 2 aperture-spacing lengths away from the closest of said two apertures.

12. The nasal CPAP canula seal of claim 11, wherein, at its furthest extent above said apertures, said nose-anchoring portion does not define an acute angle.

13. The CPAP canula seal of claim 12, wherein, at its furthest extent above said apertures, said nose anchoring portion is arcuate and does not define an angle.

14. The canula seal of claim 11, made of a sheet of hydrocolloid laminated to polyurethane.

15. The canula seal of claim 11, defining a set of cuts emanating radially from said apertures.

16. A nasal CPAP canula seal having directions of “above” and “below” by its anticipated placement on an infant face, above being higher up on said face and below being lower down, comprising:
(a) a canula supporting portion, defining two apertures for canula passage into the nostrils;
(b) a nose-anchoring portion, having a shape and extending upwardly from said apertures and designed to help anchor said nasal CPAP canula seal by adhering to the infant nose; and
(c) wherein said shape of said nose-anchoring portion includes no acute angles.

17. The canula seal of claim 16, wherein said shape of said nose-anchoring portion defines a notch, to provide a vent for condensation.

18. The canula seal of claim 17, wherein, other than in said definition of said vent, said nose-anchoring portion has an outline that is arcuate and defines no angles.

19. The canula seal of claim 16, defining a set of cuts emanating radially from said apertures.

20. The canula seal of claim 16, made of a sheet of hydrocolloid laminated to polyurethane.
